(19) United States
(12) Patent Application Publication
(10) Pub. No.: US 2025/0032152 A1
(43) Pub. Date: Jan. 30, 2025

(54) Optical Needle Guide

(71) Applicant: Bard Access Systems, Inc., Salt Lake City, UT (US)

(72) Inventors: Rachael Marie Miller, Midvale, UT (US); Matthew J. Prince, Herriman, UT (US)

(21) Appl. No.: 18/786,361

(22) Filed: Jul. 26, 2024

Related U.S. Application Data
(60) Provisional application No. 63/529,217, filed on Jul. 27, 2023.

(51) Int. Cl. A61B 17/34; A61B 8/00; A61B 8/08; A61B 90/00

(52) U.S. Cl. CPC A61B 17/3403; A61B 8/085; A61B 8/4422; A61B 8/4488; A61B 8/461; A61B 8/5223; A61B 90/08; A61B 2017/3413; A61B 2090/0807

(57) ABSTRACT

An ultrasound probe includes a light source configured to project a visual indication onto a skin surface of the patient. The visual indication includes different visual characteristics that are based on characteristics of the anatomical target such as a location with respect to the probe and/or an identification of an anatomical target as a vein or as an anatomical element other than a vein, such as an artery. Visual characteristics include shapes, locations, and/or colors of the projected visual indication. Logic of the probe performs location and/or identification processes on ultrasound image data that may include applying trained machine-learning models to the ultrasound image data. Some embodiments, include a virtual/augmented reality headset and/or a needle tracking system. An ultrasound system includes machine-learning logic that generates the trained machine-learning models from historical ultrasound image data sets and actual anatomical target location/identification data sets.

### PRIORITY

This application claims the benefit of priority to U.S. Provisional Application No. 63/529,217, filed Jul. 27, 2023, which is incorporated by reference in its entirety into this application.

### BACKGROUND

Ultrasound imaging is a widely accepted tool for guiding interventional instruments such as needles to targets such as blood vessels or organs in the human body. In order to successfully guide, for example, a needle to a blood vessel using ultrasound imaging, the needle is monitored in real-time both immediately before and after a percutaneous puncture in order to enable a clinician to determine the distance and the orientation of the needle to the blood vessel and ensure successful access thereto. Current needle guiding systems include various limitations. Mechanical needle guides used with and attached to ultrasound probes restrict needle movement. Ultrasound images displayed on a screen require viewing the screen while inserting the needle thus requiring the user to look away from the insertion site during insertion. Magnetic needle tracking system require the added expense of magnetized needles and magnetometers.

Disclosed herein are systems, devices, and methods that address these and other limitations associated with utilizing ultrasound imaging to provide guidance during vascular access procedures.

### SUMMARY

Disclosed herein is an ultrasound probe that, according to some embodiments, includes a probe head having an array of ultrasonic transducers configured to (i) emit generated ultrasound signals into a target area of a patient, (ii) receive reflected ultrasound signals from the patient, and (iii) convert the reflected ultrasound signals into corresponding electrical signals. The ultrasound probe further includes a light source configured to project a visual indication onto a skin surface of the patient and a console coupled with the probe head and the light source. The console includes a signal converter configured to convert the electrical signals into ultrasound image data including an ultrasound image of the target area, one or more processors, and a non-transitory computer-readable medium having logic stored thereon. The logic, when executed by the one or more processors, causes operations of the probe that include (i) performing a determination process on the ultrasound image data to determine when an anatomical target is present within the ultrasound image and (ii) activating the light source to project the visual indication onto the skin surface, where the visual indication includes one or more visual characteristics based on one or more characteristics of the anatomical target.

In some embodiments, the ultrasound probe includes a button configured to enable a user to selectively activate and deactivate the light source, and in some embodiments, the operations further include deactivating the light source when the anatomical target is not present within the ultrasound image.

In some embodiments, the light source includes a separate light source module attached to and operably coupled with the ultrasound probe. The separate light source module may be configured to attach to and operably couple with the ultrasound probe when a sterile barrier is covering the probe, where the sterile barrier is disposed between the separate light source module and the ultrasound probe. The separate light source module may be wirelessly coupled with the ultrasound probe, and the separate light source module may be configured for single use.

In some embodiments, the one or more visual characteristics include at least one of a dot a line, and/or a number of colors. In some embodiments, the one or more visual characteristics include the line and the line may extend away from the ultrasound probe in a direction perpendicular to a front face of the ultrasound probe. In some embodiments, the one or more characteristics of the anatomical target include an identity of the anatomical target and/or a location of the anatomical target with respect to the ultrasound probe.

In some embodiments, the operations further include performing a location process on the ultrasound image data to determine the location of the anatomical target with respect to the ultrasound probe, and in some embodiments, activating the light source includes projecting the visual indication onto the skin surface at a location above the anatomical target. In some embodiments, the location of the visual indication defines an optimal or preferred insertion site for a needle to access the anatomical target.

In some embodiments, the operations further include performing an identification process on the ultrasound image data to identify the anatomical target as a vein or as an anatomical element other than a vein. In some embodiments, the one or more visual characteristics include a first color when the identification process identifies the anatomical target as a vein, and the one or more visual characteristics include a second color different from the first color when the identification process identifies the anatomical target as the anatomical element other than a vein.

In some embodiments, the one or more visual characteristics include a third color when the location process determines that the anatomical target is centrally located with respect to the ultrasound probe, and the one or more visual characteristics include a fourth color different from the third color when the location process determines that the anatomical target is located away from a center of the ultrasound probe.

In some embodiments, performing the location process includes applying a first trained machine-learning model to the ultrasound image data resulting in the determination of the location of the anatomical target with respect to the ultrasound probe. In some embodiments, performing the identification process includes applying a second trained machine-learning model to the ultrasound image data resulting in the identification of the anatomical target as a vein or as an anatomical element other than a vein.

In some embodiments, the ultrasound probe is operably coupled with a needle tracking system configured to determine a location and an orientation of a trackable needle with respect to the ultrasound probe, where the operations further include receiving needle tracking data from the needle tracking system and performing a tracking process on the ultrasound image data in combination with the needle tracking data to determine a location of the trackable needle with respect to the anatomical target. In such embodiments, the one or more visual characteristics include visual characteristics based on the location of the trackable needle with respect to the anatomical target. The visual characteristics based on the location of the trackable needle may be configured to indicate when the trackable needle is aligned with the anatomical target. In some embodiments, visual characteristic based on the location of the trackable needle include (i) a fifth color when the tracking process determines that the trackable needle is not aligned with the anatomical target and (ii) a sixth color different from the fifth color when the tracking process determines that the trackable needle is aligned with the anatomical target.

Also disclosed herein is an ultrasound system that includes an ultrasound probe according to any of the embodiments described above except where the ultrasound probe is coupled with a headset (e.g., an augmented or virtual reality headset) in leu of the light source.

Also disclosed herein is a computerized method that, according to some embodiments, includes receiving ultrasound image data converted from electrical signals generated by an ultrasound probe head of an ultrasound probe, where the ultrasound probe head is placed on a skin surface of a patient over a target area, and where the ultrasound probe head includes an array of ultrasonic transducers configured to (i) emit generated ultrasound signals into a target area of a patient, (ii) receive reflected ultrasound signals from the patient, and (iii) convert the reflected ultrasound signals into corresponding electrical signals. The method further includes performing a determination process on the ultrasound image data to determine when an anatomical target is present within the ultrasound image and activating the light source of the ultrasound probe to project a visual indication onto the skin surface, where the visual indication includes one or more visual characteristics based on one or more characteristics of the anatomical target.

In some embodiments, the method further includes performing a location process on the ultrasound image data to determine the location of the anatomical target within the target area with respect to the ultrasound probe, where activating the light source further includes projecting the visual indication onto the skin surface at a location above the anatomical target, and in some embodiments, performing the location process includes applying a first trained machine-learning model to the ultrasound image data resulting in the determination of the location of the anatomical target with respect to the ultrasound probe.

In some embodiments, the method further includes performing an identification process on the ultrasound image data to identify the anatomical target as a vein or as an anatomical element other than a vein, where activating the light source further includes at least one of (i) projecting the visual indication having a first color when the identification process identifies the anatomical target as a vein or (ii) projecting the visual indication having second color, different from the first color, when the identification process identifies the anatomical target as the anatomical element other than a vein, and in some embodiments, performing the identification process includes applying a second trained machine-learning model to the ultrasound image data resulting in the identification of the anatomical target as the vein or as the anatomical element other than a vein.

Also disclosed herein is a ultrasound imaging system that, according to some embodiments, includes a plurality of ultrasound probes, where each ultrasound probe includes a probe head having an array of ultrasonic transducers configured to (i) emit generated ultrasound signals into a target area of a patient, (ii) receive reflected ultrasound signals from the patient, and (iii) convert the reflected ultrasound signals into corresponding electrical signals. Each ultrasound probe includes further includes a light source configured to project a visual indication onto a skin surface of the patient and a console coupled with the probe head and the light source. The console includes a signal converter configured to convert the electrical signals into ultrasound image data including an ultrasound image of the target area. The console further includes one or more processors and a non-transitory computer-readable medium having logic stored thereon. The logic, when executed by the one or more processors, causes operations of the probe that include (i) performing a location process on the ultrasound image data to determine a location of the anatomical target with respect to the ultrasound probe, where performing the location process includes applying a first trained machine-learning (ML) model to the ultrasound image data and (ii) activating the light source to project the visual indication onto the skin surface at a location above the anatomical target. The system further includes a computing system coupled with each of the plurality of ultrasound probes, where the computing system includes a non-transitory computer-readable medium having ML logic stored thereon. The ML logic, when executed by processors, performs ML operations that include performing a first ML algorithm on historic ultrasound image data sets to define the first trained ML model. The historical ultrasound image data sets include anatomical target location data sets received from the ultrasound probes and actual anatomical target location data sets, and each actual anatomical target location data set corresponds to an anatomical target location data set in a one-to-one relationship.

In some embodiments of the system, the operations further include performing an identification process on the ultrasound image data to determine an identity of the anatomical target as a vein or as an anatomical element other than a vein, and performing the identification process includes applying a second trained ML model to the ultrasound image data. The operations further include (i) activating the light source to project the visual indication having a first color when the identity of the anatomical target includes a vein and/or (ii) activating the light source to project the visual indication having a second color when the identity of the anatomical target includes the anatomical element other than a vein, where the second color is different from the first color. The ML operations further include performing a second ML algorithm on the historic ultrasound image data sets to define the second trained ML model, where the historical ultrasound image data sets further include anatomical target identification data sets received from the ultrasound probes and actual anatomical target identification data sets, and where each actual anatomical target identification data set corresponds to an anatomical target identification data set in a one-to-one relationship.

These and other features of the concepts provided herein will become more apparent to those of ordinary skill in the art in view of the accompanying drawings and following description, which describe particular embodiments of such concepts in greater detail. Further details and features of the concepts provided here may be disclosed in one or more of U.S. Pat. No. 10,322,230 and U.S. Published Application No. 2021-0085282, each of which is incorporated by reference in its entirety into this application.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 illustrates an ultrasound probe in contact with a skin surface of a patient, according to some embodiments;

FIG. 2A is an illustration of the ultrasound probe of FIG. 1 projecting via a light source a first visual indication onto the skin surface, according to some embodiments;

FIG. 2B is an illustration of the ultrasound probe of FIG. 1 projecting via the light a second visual indication onto the skin surface, according to some embodiments;

FIG. 3 illustrates a block diagram of a console of the ultrasound probe of FIG. 1, according to some embodiments;

FIG. 4 illustrates a block diagram of a computerized method of the ultrasound probe of FIG. 1, according to some embodiments;

FIG. 5 illustrates an ultrasound system for defining trained machine-learning modules for the ultrasound probe of FIG. 1, according to some embodiments;

FIG. 6 illustrates another embodiment of an ultrasound probe where the light source is a separate component, according to some embodiments;

FIG. 7 illustrates another embodiment of the ultrasound probe further including a headset, according to some embodiments; and

FIG. 8 illustrates another embodiment of the ultrasound probe further including needle tracking system, according to some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

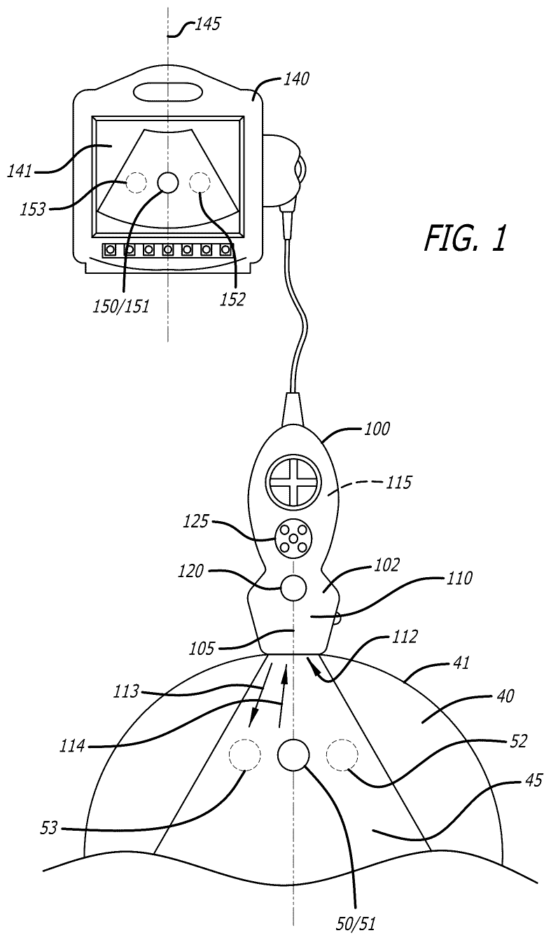

FIG. 1 illustrates an ultrasound probe in contact with a skin surface of a patient, according to some embodiments. The ultrasound probe (probe) 100 includes a probe head 110 having an array of ultrasound transducers 112 disposed along a patient contact surface thereof. The ultrasound transducers 112 are configured to (i) project ultrasound signals 113 into the patient 40, (ii) receive reflected ultrasound signals 114 from the patient 40, and (iii) convert the reflected ultrasound signals 114 into electrical signals. The array of ultrasound transducers 112 may be configured to detect motion of the anatomical target 50 such as pulsing of the blood vessel wall or motion of blood within a blood vessel. The probe 100 includes a console 115 that is generally configured to generate ultrasound image data from the electrical signals as further described below. The probe 100 is placed on the patient 40 so that the probe head 110 is positioned over a target area 45 of the patient 40. Logic of the console 115 is configured to detect the presence of one or more anatomical targets within the target area 45, such as the anatomical target 50, for example. As illustrated, in some instances, the probe 100 may be positioned on the patient 40 such that the anatomical target 50 is centrally located with respect to the probe 100, i.e., so that the anatomical target 50 is located at position 51 which is aligned with a central axis 105 of the probe 100. In other instances, the probe 100 may be located at positions spaced away on either side from the central axis 105, such as the right position 52 or the left position 53.

Although not required, the probe 100 may be coupled, via a wired or wireless connection, with a display 140 so that an ultrasound image 141 as defined by the ultrasound image data may be depicted on the display 140. In the illustrated embodiment, the ultrasound image 141 depicts an anatomical target image 150 of the anatomical target 50. As shown, the anatomical target image 150 is centrally located within the ultrasound image 141 (i.e., the position 151 of the anatomical target image 150 is aligned with a central axis 145 of the ultrasound image 141) consistent with the central location of the anatomical target 50 with respect to the probe 100. As also shown, the anatomical target image 150 may be depicted at locations 152 or 153 with respect to the central axis 145 consistent with the respective positions 52 or 53 of the anatomical target 50 with respect to the probe 100.

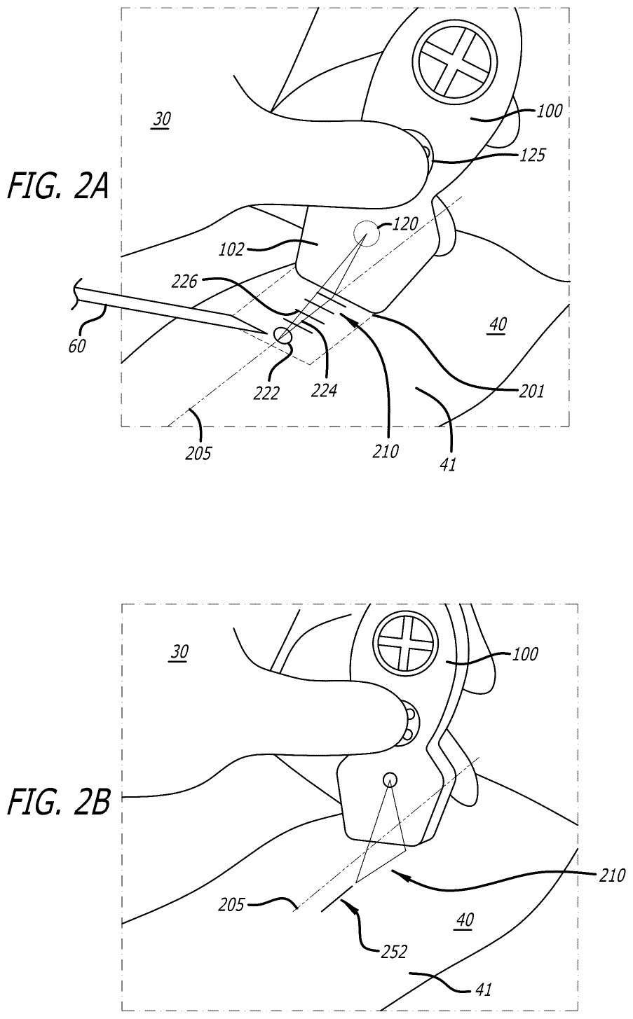

The probe 100 further includes a light source 120 configured to project a visual indication onto the skin surface as described further in relation to FIGS. 2A-2B. The probe 100 may also include one or more buttons 125 configured to enable a user to operate the probe 100, including activating and/or deactivating the light source 120. The light source 120 may include any suitable light emitting device, such as a laser, a light emitting diode, or an optical fiber for example. Further, the light source 120 may include any number (e.g., 1, 2, 3, or more) of light emitting devices. In the illustrated embodiment, the light source 120 may be located on a front face 102 of the probe 100. However, in the other embodiments, the light source 120 may be located at other positions on the probe 100, including multiple positions, such as on the right side, left side, or back side of the probe 100.

FIG. 2A illustrates the probe 100 projecting, via the light source 120, a visual indication 210 onto the skin surface 41 of the patient 40, according to one embodiment. The visual indication 210 may be configured to convey information to the user 30 based on a number of characteristics of the anatomical target 50. According to one embodiment, the visual indication 210 may indicate that the probe 100 has detected the presence of the anatomical target 50 within the target area 45 (see FIG. 1). In such an embodiment, the visual indication 210 may be projected (i.e., the light source 120 may be activated) only when the anatomical target 50 is detected within the target area 45. Said another way, the activation of the light source 120 may be prevented unless the anatomical target 50 detected within the target area 45. In some embodiments, the visual indication 210 may include an illumination of an area 201 in front of the probe 100 to indicate the detection of the anatomical target 50 within the target area 45. According to one instance of use, the user 30 may adjust the position of the probe 100 on the skin surface until the light source 120 activated illuminating the area 201 as a result of detecting the anatomical target 50 within the target area 45.

The visual indication 210 may include a shape such as a line 224 or a dot 222 configured to indicate a location on the skin surface 41. In some embodiments, the line 224 or a dot 222 may be projected in alignment with a second central axis 205 of the probe 100, where the second central axis 205 (i) intersects the central axis 105 shown in FIG. 1 and (ii) extends perpendicularly away from the front face 102. In such embodiments, the probe 100 may determine that the anatomical target 50 is located at the position 51 (see FIG. 1). As such, the line 224 or a dot 222 may be projected directly over the anatomical target 50. According to another instance of use, the user 30 may adjust the position of the probe 100 on the skin surface 45 until the central axis 105 (see FIG. 1) is disposed over the anatomical target 50, at which point the visual indication 210 may include the line 224 and/or the dot 222.

When the visual indication 210 includes the line 224, the line 224 may indicate the presence of the anatomical target 50 directly beneath the line 224. As such, the user 30 may be confident that a needle 60 inserted into the patient along the line 224 will intersect the anatomical target 50. Similarly, when the visual indication 210 includes the dot 225, the dot 225 may indicate the presence of the anatomical target 50 directly beneath the dot 225. As such, the user 30 may be confident that a needle 50 inserted into the patient at the dot 225 will intersect the anatomical target 50. In some embodiment, the dot 225 may be projected at a defined distance from the front face 102 to indicate an optimal or preferred insertion site for the needle 50. In some embodiments, the visual indication 210 may include a set of graduation lines 226 (or other indicium) that indicate defined distances from the front face 102, such as 0.5 cm, 1 cm, 1.5 cm, and 2 cm, for example. Of course, other distances may be indicated by the graduation lines 226 as may be contemplated by one of ordinary skill.

The visual indication 210 may also include a number of colors to indicate characteristics of the anatomical target 50. In some embodiments, a characteristic of the anatomical target 50 may include an identity. In the illustrated embodiment, the logic may determine the identity of the anatomical target 50 as a blood vessel and may further identify the blood vessel as a vein or some other anatomical element including an artery. As such, the visual indication 210 may also include a color or some other visual characteristic in accordance with the identity of the anatomical target 50. According to one embodiment, the logic may determine that the anatomical target 50 is a vein and project the visual indication 210 having a first color (e.g., green). Similarly, the logic may determine that the anatomical target 50 is an anatomical element other than a vein (e.g., an artery) and project the visual indication 210 having a second color (e.g., red) that is different from the first color.

According to another embodiment, the logic may determine that the anatomical target 50 is located beneath the line 224 (i.e., centrally located with respect to the ultrasound probe) and project the visual indication 210 having a third color. Similarly, the logic may determine that the anatomical target 50 is located at a position spaced away from the line 224 and project the visual indication 210 having a fourth color that is different from the third color.

In some instances of use, the user may deploy the probe 100 to find a vein to be accessed by the needle 60. In such an instances, the user may adjust the position of the probe 100 on the skin surface until the probe 100 projects the visual indication having the third color, in which case the user may have confidence that the needle 60, when inserted into the patient 40 along the line 224 or at the dot 222, will intersect the vein.

Other visual characteristics of the visual indication 210 are also considered as may be contemplated by one of ordinary skill, such as a blinking or flashing light, color variation, textual messages, indicia, shapes, light intensity, or multiple projections, for example, to indicate the characteristics of the anatomical target 50 described above, or other characteristics such as case of access, depth from the skin surface 41, or the presence of an obstruction, for example.

FIG. 2B illustrates the probe 100 projecting the visual indication 210 onto the skin surface 41 of the patient 40 according to another embodiment, where the anatomical target 50 is located at a position that is offset from the second central axis 205 such as at the position 52 or the position 53 as shown in FIG. 1. In the instance shown in FIG. 2B, the anatomical target 50 is located at the position 52. However, the description that follows may also apply to an instance where the anatomical target 50 is located at the position 53. In accordance with this embodiment, the characteristics of the anatomical target 50 include the location of the anatomical target 50 with respect to the probe 100. As shown, the visual indication 210 is projected onto the skin surface 41 at a position 252 offset from the second central axis 205 to indicate that the anatomical target 50 is located at the position 52 which is offset from the central axis 105 (see FIG. 1).

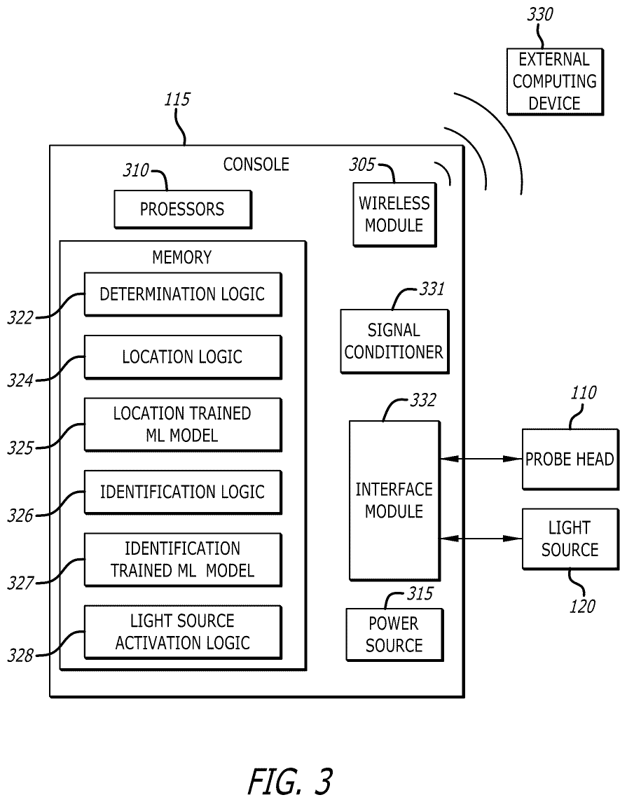

FIG. 3 illustrates a block diagram of the console 115, according to some embodiments. The console 115 is generally configured to govern the operation of the probe 100. The console 115 includes one or more processors 310 and a memory 320 (e.g., a non-transitory computer-readable medium) having logic stored thereon. The logic includes determination logic 322, location logic 324, identification logic 326, and light source activation logic 328. The console 115 is powered via a power source 315 (e.g., a battery). The console 115 may optionally include a wireless module 305 to facilitate wireless communication with the external computing device 330 (sometimes referred to as a computing system) as further described below.

The console 115 includes an interface module 332 (e.g., a connector set) configured to enable operative coupling of the console 115 with the probe head 110 and/or the light source 120. A signal conditioner 331 converts electrical signals from the probe head 110 to ultrasound image data for processing by the one or more processor 310 according to the logic. Similarly, the signal conditioner 331 converts digital data from the processors 310 to electrical signals for the probe head 110 and/or the light source 120.

The determination logic 322 receives ultrasound image data from the probe head 110 and performs a determination process on the ultrasound image data to detect/determine the presence of the anatomical target 50 within the target area 45. Upon detection of the anatomical target 50, the location logic 324 performs a location process on the ultrasound image data to determine the position of the anatomical target 50 with respect to the probe 100, such as at the positions 51, 52, or 53, for example.

Further upon detection of the anatomical target 50, the identification logic 326 may perform an identification process on the ultrasound image data to identify the anatomical target 50, i.e., determine if the anatomical target 50 is a vein or is some other anatomical element, such as a bone, a cluster of nerves, an artery, or a bifurcation of a blood vessel, for example. According to one embodiment, the ultrasound image data may include doppler ultrasound data and the identification logic 326 may be configured to identify the anatomical target 50 based at least partially on the doppler ultrasound data, where the doppler ultrasound data is configured to detect/determine a motion of the anatomical target 50 or portion thereof. Such motion may include pulsing of at least a portion of the anatomical target 50 or a flow of blood within the anatomical target 50.

According to one embodiment, the memory 320 may optionally include a location trained machine-learning (ML) model 325 and performing the location process on the ultrasound image data may include applying the location trained ML model 325 to the ultrasound image data. A result of the applying the location trained ML model 325 to the ultrasound image data may include the determination of the location of the anatomical target 50 with respect to the probe 100.

According to one embodiment, the memory 320 may optionally include an identification trained machine-learning (ML) model 327 and performing the identification process on the ultrasound image data may include applying the identification trained ML model 327 to the ultrasound image data. A result of the applying the identification trained ML model 327 to the ultrasound image data may include the determination of the identity of the anatomical target 50 as a vein, or some other anatomical element, such as an artery, for example.

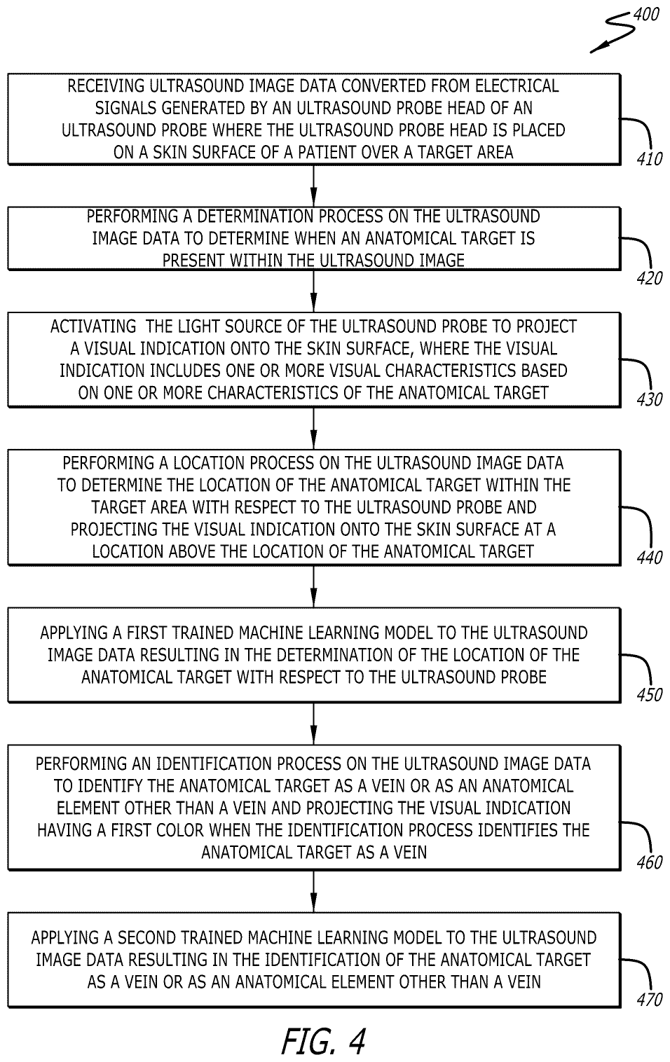

FIG. 4 is a block diagram of a computerized method 400 that, according to some embodiments, includes all or any subset of the following actions, operations, or processes. Each block illustrated in FIG. 4 represents an operation of the method 400 performed by the ultrasound probe disclosed herein. The method 400 includes receiving ultrasound image data converted from electrical signals generated by an ultrasound probe head of an ultrasound probe where the ultrasound probe head is placed on a skin surface of a patient over a target area (block 410). The ultrasound probe head includes an array of ultrasonic transducers configured to (i) emit generated ultrasound signals into a target area of a patient, (ii) receive reflected ultrasound signals from the patient, and (iii) convert the reflected ultrasound signals into corresponding electrical signals. The method 400 may further include performing a determination process on the ultrasound image data to determine when an anatomical target is present within the ultrasound image (block 420). The method 400 may further include activating the light source of the ultrasound probe to project a visual indication onto the skin surface, where the visual indication includes one or more visual characteristics based on one or more characteristics of the anatomical target (block 430).

The method 400 may further include performing a location process on the ultrasound image data to determine the location of the anatomical target within the target area with respect to the ultrasound probe and projecting the visual indication onto the skin surface at a location above the anatomical target (block 440). The method 400 may further include applying a first trained machine-learning model to the ultrasound image data resulting in the determination of the location of the anatomical target with respect to the ultrasound probe (block 450).

The method 400 may further include performing an identification process on the ultrasound image to identify the anatomical target as a vein or some other anatomical element and projecting the visual indication having a first color when the identification process identifies the anatomical target as a vein (block 460). The method 400 may further include projecting the visual indication having second color, different from the first color, when the identification process identifies the anatomical target as an anatomical element other than a vein, including an artery. The method 400 may further include applying a second trained machine-learning model to the ultrasound image data resulting in the identification of the anatomical target as a vein or some other anatomical element (block 470).

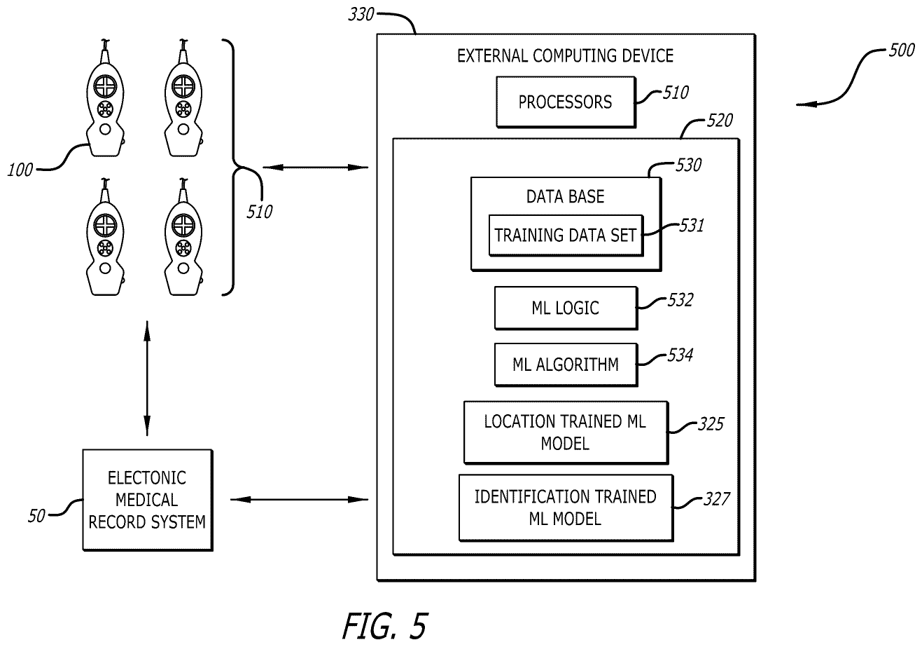

FIG. 5 illustrates an ultrasound imaging system (system) 500, according to some embodiments. The system 500 is generally configured to define the location trained ML model 325 and/or the identification trained ML model 327. The system 500 generally includes a plurality of probes 510 (i.e., multiple probes 100) coupled with the external computing device 330. According to one embodiment, the external computing device 330 may include a network server. In some embodiments, the external computing device 330 may be coupled with or incorporated into the EMR system 550. In other embodiments, the external computing device 330 may be incorporated into one or more of the probes 100. The plurality of probes 510 may be wirelessly coupled with the EMR system 550 and in such embodiments, the plurality of probes 510 may transmit data such as the historical ultrasound image data sets to the EMR system 550.

The external computing device 330 includes a database 530 and machine-learning (ML) logic 532 stored in memory 510 (e.g., a non-transitory computer-readable medium). The ML logic 532 is configured to acquire historical ultrasound image data sets from the plurality of probes 510 and/or the EMR system 550 to form a training data set 531 stored in the data base 530. The ML logic 532 is further configured to apply an ML algorithm 534 to the training data set 531 to define the location trained ML model 325 and/or the identification trained ML model 327, where the ML logic 532 may be composed of or configured to execute a plurality of ML algorithms 534 (e.g., predictive algorithms such as linear regression, logistic regression, classification and regression trees, Naïve Bayes, K-Nearest neighbors, etc.). The historical ultrasound image data sets include location data sets and/or identification data sets received from the plurality of probes 510 and actual anatomical target data sets that correspond individually (i.e., according to one-to-one relationship) to the ultrasound image data sets. More specifically each ultrasound image data set corresponds with an actual anatomical target data set for a single ultrasound imaging event.

The location data set for the ultrasound imaging event includes the determined location of the anatomical target, i.e., the location of the anatomical target 50 with respect to the probe 100 such as one of the positions 51-53 (see FIG. 1) as determined by the probe 100. The actual anatomical target data set includes an independent determination of the location, such as a visual determination of the location of the anatomical target image 150 as depicted on the display 140 or a direct determination by the user 30 such as a location of the needle 60 once inserted with respect to probe 100. In some instances, the independent determination of the location may be recorded in the EMR for the patient 40. Similarly, the actual anatomical target data set may include an independent identification of the anatomical target 50 as a vein or some other anatomical element, including an artery. The independent identification may include the utilization of a separate ultrasound imaging system, a needle tracking system, a catheter tracking system, or the like. In some instances, the independent identification of the anatomical target 50 may be recorded in the EMR for the patient 40.

The external computing device 330 may be coupled with the EMR system 550, and the ML logic 532 may acquire the actual anatomical target data sets from the EMR system 550. The location trained ML model 325 and/or the identification trained ML model 327 may be stored in the memory 520 of the external computing device 330. The ML logic 532 may transmit or communicate location trained ML model 325 and/or the identification trained ML model 327 to the probes 100 for storage in the memory 320.

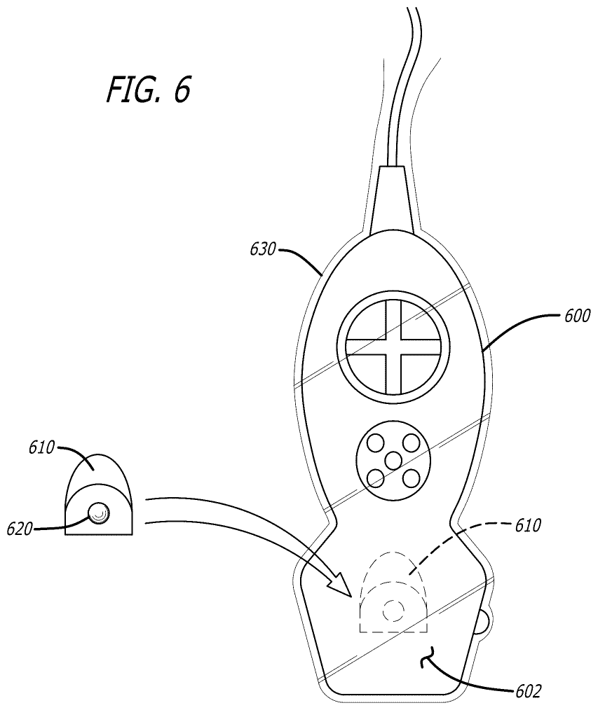

FIG. 6 illustrates another embodiment of an ultrasound probe 600 that can, in certain respects, resemble components, features and functionalities of the ultrasound probe 100 described in connection with FIGS. 1-4. It will be appreciated that all the illustrated embodiments may have analogous features. Relevant disclosure set forth above regarding similarly identified features thus may not be repeated hereafter. Moreover, specific features of the ultrasound probe 100 and related components shown in FIGS. 1-4 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the ultrasound probe 600. Any suitable combination of the features, and variations of the same, described with respect to the ultrasound probe 100 and components illustrated in FIGS. 1-4 can be employed with the ultrasound probe 600 system and components of FIG. 6, and vice versa. This pattern of disclosure applies equally to further embodiments depicted in subsequent figures and described hereafter.

The ultrasound probe (probe) 600 includes a light source module 610 that is a separate component from the probe 600. The light source module 610 is attachable to the probe 600. In the illustrated embodiments, the light source module 610 is configured to attach to the front face 602 of the probe 600. However, in other embodiments, the light source module 610 may be attached to probe 600 at other locations, such as the right side, left side or back side, for example. The light source module 610 may also be detachable from the probe 600. In some embodiments, the light source module 610 may be configured for single use, i.e., the light source module 610 may be a disposable component. The light source module 610 includes the light source 620. The light source module 610 may be attached to the probe 600 via any suitable fashion, such as via a strap, a clip, a clamp, an adhesive, or one or more magnets, for example.

The light source module 610 is configured to operably coupled with the probe 600 when the light source module 610 is attached to the probe. Although, in some embodiments, the light source module 610 may operably couple with the probe 600 even when the light source module 610 is not physically attached to the probe 600. In some embodiments, the light source module 610 may include a number of electrical connecting members (e.g., pins) configured to make electrical contact with corresponding electrical connecting members (e.g., sockets) of the probe 600.

According to one embodiment, the light source module 610 may be configured to wirelessly couple with the probe 600. As such, the light source module 610 may include console components, such as a battery, a processor, memory, and a wireless module, for example to enable the light source module 610 to operably couple with the probe 600.

In some embodiments, the probe 600 may include a sterile barrier 630, such as a plastic or elastomeric covering (e.g., a bag) that covers the probe 600 including the front face 602. In such embodiments, the light source module 610 may attach to the probe 600, where the sterile barrier 630 is disposed between the light source module 610 and the probe 600. In other words, the light source module 610 is configured to attach to the probe 600 without disabling the sterile barrier 630.

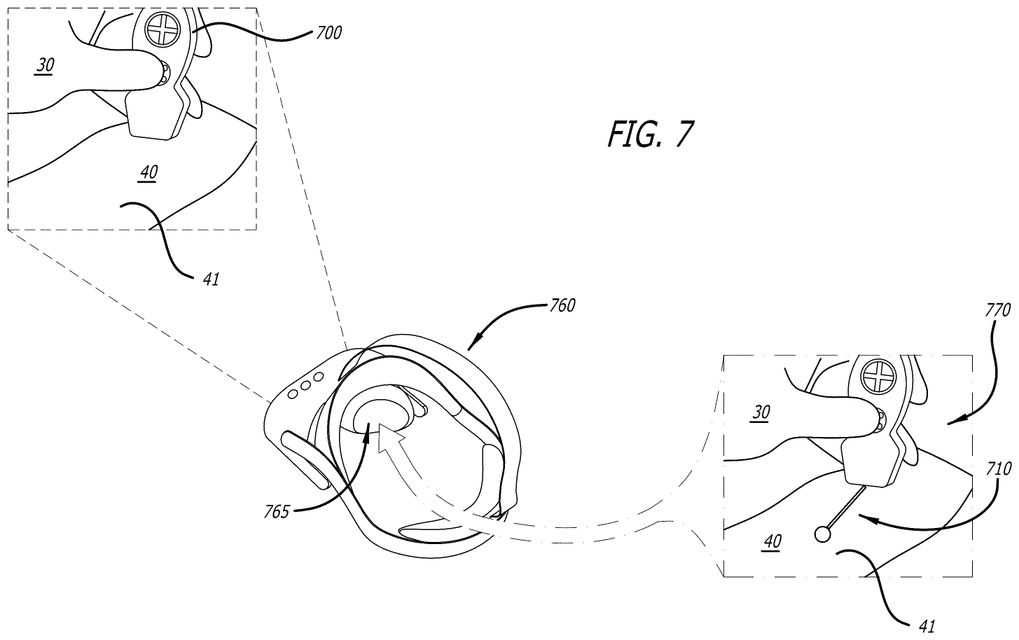

FIG. 7 illustrates an embodiment of an ultrasound probe (probe) 700 coupled with a headset 760. The headset 760 may be a virtual or augmented reality headset configured to depict on a display 765 an image 770 of the probe 700 in use with the patient 40. In some embodiments, the probe 700 may omit the light source. As such, the image 770 may include the visual indication 710 appearing on the skin surface 41 of the patient 40. The visual indication 710 may include all or any subset of the features of the visual indication 210 described in relation to FIGS. 2A-2B. The headset 760 may be coupled with the probe 700 via a wired or wireless connection.

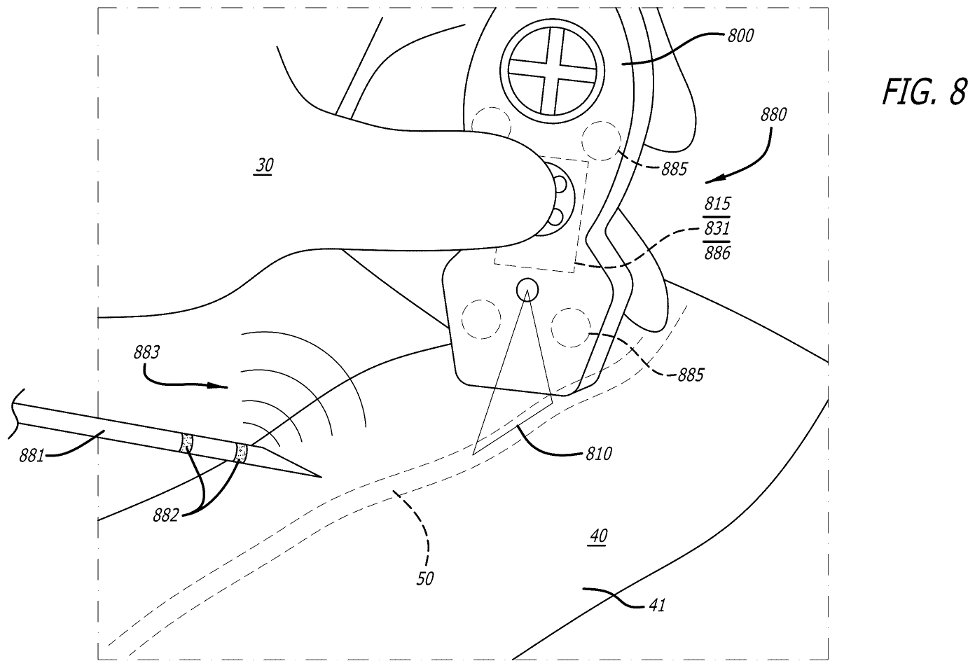

FIG. 8 illustrates an embodiment of an ultrasound probe (probe) 800 having a needle tacking system 880 integrated into or otherwise operably coupled with the probe 800. The needle tacking system 880 is generally configured to track a trackable needle 881 with respect to the anatomical target 50. More specifically, the probe 800 determines the location of the anatomical target 50 with respect to the probe 800 and the needle tacking system 880 determines the location of the trackable needle 881 with respect to the probe 800.

The needle tacking system 880 is configured to magnetically track the trackable needle 881. The trackable needle 881 includes a number (e.g., 1, 2, 3 or more) of magnetic elements 882 configured to generate one or more magnetic fields 883. The needle tacking system 880 further includes a number (e.g., 1, 2, 3, or more) of magnetometers 885 configured to detect the one or more magnetic fields 883. In the illustrated embodiment, the console 815 may in some respects resemble the components and features of the console 115 of FIG. 3. A signal conditioner 831 includes the features and functionalities of the signal conditioner 331 and is further configured to receive electrical tracking signals from the magnetometers 885 and convert the electrical tracking signals into needle tracking data. The console 815 includes tracking logic 886 configured to receive the needle tracking data. The tracking logic 886 performs a tracking process on the ultrasound image data in combination with the needle tracking data to determine a location of the trackable needle 881 with respect to the anatomical target 50.

A visual indication 810 may include all or any subset of the features of the visual indication 210 and may further include one or more visual characteristics based on the location of the trackable needle 881 with respect to the anatomical target 50. In the illustrated embodiment, the visual characteristics are configured to indicate when the trackable needle 881 is aligned with the anatomical target 50. More specifically, the visual characteristics based on the location of the trackable needle 881 with respect to the anatomical target 50 are configured to indicate when a location and orientation of the trackable needle 881 with respect to the anatomical target 50 are such that insertion of the trackable needle 881 into the patient 40 will enter or intersect the anatomical a target 50. In some embodiments, the visual characteristics based on the location of the trackable needle 881 include (i) a fifth color (e.g., red) when the tracking process determines that the trackable needle is not aligned with the anatomical target and (ii) a sixth color (e.g., green) different from the fifth color when the tracking process determines that the trackable needle is aligned with the anatomical target.

Further details regarding the needle tracking system 880 can be found in the following U.S. patent and patent application publications 2014/0257080; 2014/0257104; U.S. Pat. Nos. 9,155,517; 9,257,220; 9,459,087; and 9,597,008, each of which is incorporated by reference in its entirety into this application.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An ultrasound probe, comprising:
a probe head including an array of ultrasonic transducers configured to emit generated ultrasound signals into a target area of a patient, receive reflected ultrasound signals from the patient, and convert the reflected ultrasound signals into corresponding electrical signals;
a light source configured to project a visual indication onto a skin surface of the patient; and
a console coupled with the probe head and the light source, the console including a signal converter configured to convert the electrical signals into ultrasound image data including an ultrasound image of the target area, one or more processors, and a non-transitory computer-readable medium having stored thereon logic that, when executed by the one or more processors, causes operations including:
performing a determination process on the ultrasound image data to determine when an anatomical target is present within the ultrasound image; and
activating the light source to project the visual indication onto the skin surface, the visual indication including one or more visual characteristics based on one or more characteristics of the anatomical target.

2. The probe according to claim 1, wherein the light source includes a separate light source module attached to and operably coupled with the ultrasound probe.

3. The probe according to claim 2, wherein the separate light source module is configured to attach to and operably couple with the ultrasound probe having a sterile barrier covering the probe, the sterile barrier disposed between the separate light source module and the ultrasound probe.

4. The probe according to claim 2, wherein the separate light source module is wirelessly coupled with the ultrasound probe.

5. The probe according to claim 2, wherein the separate light source module is configured for single use.

6. The probe according to claim 1, wherein the operations further include deactivating the light source when the anatomical target is not present within the ultrasound image.

7. The probe according to claim 1, wherein the one or more visual characteristics includes at least one of a dot or a line.

8. The probe according to claim 7, wherein the one or more visual characteristics include the line, and wherein the line is configured to extend away from the ultrasound probe in a direction perpendicular to a front face of the ultrasound probe.

9. The probe according to claim 1, wherein the one or more characteristics of the anatomical target include at least one of an identity of the anatomical target or a location of the anatomical target with respect to the ultrasound probe.

10. The probe according to claim 9, wherein the operations further include performing a location process on the ultrasound image data to determine the location of the anatomical target with respect to the ultrasound probe.

11. The probe according to claim 10, wherein activating the light source includes projecting the visual indication onto the skin surface at a location above the anatomical target.

12. The probe according to claim 11, wherein the location of the visual indication defines an insertion site for a needle to access the anatomical target.

13. The probe according to claim 9, wherein the operations further include performing an identification process on the ultrasound image data to identify the anatomical target as a vein or as an anatomical element other than a vein.

14. The probe according to claim 13, wherein the one or more visual characteristics include a number of colors.

15. The probe according to claim 14, wherein the one or more visual characteristics include:
a first color when the identification process identifies the anatomical target as a vein; and
a second color, different from the first color, when the identification process identifies the anatomical target as the anatomical element other than a vein.

16. The probe according to claim 15, wherein the one or more visual characteristics include:
a third color when the location process determines that the anatomical target is centrally located with respect to the ultrasound probe; and
a fourth color different from the third color when the location process determines that the anatomical target is located away from a center of the ultrasound probe.

17. The probe according to claim 10, wherein performing the location process includes applying a first trained machine-learning model to the ultrasound image data resulting in the determination of the location of the anatomical target with respect to the ultrasound probe.

18. The probe according to claim 13, wherein performing the identification process includes applying a second trained machine-learning model to the ultrasound image data resulting in the identification of the anatomical target as the vein or as the anatomical element other than a vein.

19. The probe according to claim 10, wherein:
the ultrasound probe is operably coupled with a needle tracking system configured to determine a location and an orientation of a trackable needle with respect to the ultrasound probe, the operations further including:
receiving needle tracking data from the needle tracking system; and
performing a tracking process on the ultrasound image data in combination with the needle tracking data to determine a location of the trackable needle with respect to the anatomical target, and
the one or more visual characteristics include visual characteristics based on the location of the trackable needle with respect to the anatomical target.

20. The probe according to claim 19, wherein the visual characteristics based on the location of the trackable needle are configured to indicate when the trackable needle is aligned with the anatomical target.

21. The probe according to claim 20, wherein the visual characteristics based on the location of the trackable needle include:
a fifth color when the tracking process determines that the trackable needle is not aligned with the anatomical target; and
a sixth color different from the fifth color when the tracking process determines that the trackable needle is aligned with the anatomical target.

22-44. (canceled)
